(19) United States
(12) Patent Application Publication
(10) Pub. No.: US 2012/0128767 A1
(43) Pub. Date: May 24, 2012

(54) THERAPEUTIC CALCIUM PHOSPHATE PARTICLES AND METHODS OF MAKING AND USING SAME

(76) Inventors: William W. LEE, San Diego, CA (US); Feng LU, Shanghai (CN)

(21) Appl. No.: 12/434,557

(22) Filed: May 1, 2009

Related U.S. Application Data
(60) Provisional application No. 61/049,627, filed on May 1, 2008.

(51) Int. Cl. A61K 9/48; A61K 9/28; A61K 38/02; A61K 31/715; A61K 31/7088; A61K 31/70; A61K 38/28; A61K 38/18; A61K 38/27; A61K 38/29; A61K 38/23; A61K 38/22; A61K 47/04; A61K 38/21; C01B 25/32; A61P 3/10; A61P 3/06; A61P 9/00; A61P 3/04; A61P 9/04; A61P 9/10; A61P 9/06; A61P 9/12; A61K 9/14; B82Y 5/00

(52) U.S. Cl. 424/452; 424/499; 424/474; 424/490; 514/1.1; 514/54; 514/44 R; 514/23; 514/5.9; 514/7.7; 514/11.3; 514/11.8; 514/11.9; 514/9.7; 514/769; 424/463; 424/465; 424/85.4; 423/311; 977/773; 977/906

(57) ABSTRACT

The present invention provides novel calcium phosphate nanoparticles suitable for efficient encapsulation of biologically active molecules. The invention further provides pharmaceutical compositions comprising these nanoparticles, as well as methods of making such nanoparticles and using them as carriers for therapeutic delivery of biologically active macromolecules.

### CROSS-REFERENCE TO RELATED APPLICATIONS

This application claims the benefit of U.S. Ser. No. 61/049,627, filed May 1, 2008, which is incorporated herein by reference in its entirety.

### FIELD OF THE INVENTION

The present invention generally relates to the field of drug delivery. More specifically, the invention relates to novel calcium phosphate particles suitable for efficient encapsulation of biologically active molecules. The invention also relates to pharmaceutical compositions comprising these particles, as well as methods of making such particles and using them as carriers for therapeutic delivery of biologically active macromolecules.

### BACKGROUND OF THE INVENTION

Macromolecule pharmaceuticals, including proteins, peptide, polysaccharide, nucleic acid, lipids or the combination, are an increasingly important class of drugs to treat various medical conditions. The primary route for administrating macromolecular pharmaceuticals is hypodermal injection, which is unpleasant, expensive and often results in poor compliance. Oral delivery is a preferred route to administer medicine. However, macromolecular drugs are poorly absorbed through intestines and can be easily destroyed by stomach acid and particularly by degrading enzymes in gastrointestinal tract. A promising approach to overcome the barriers for oral macromolecule delivery is to use nanoparticles, which offer protection against degradation and enhance intestinal absorption.

It has been reported that nanoparticles loaded with insulin can be used to deliver bioactive insulin to animals. For example, prevention of plasma glucose elevation by insulin entrapped in poly(lactide-co-glycolide) nanoparticles with fumaric anhydride oligomer and iron oxide additives was observed by Carino et al., (J. Controlled Release 65:261, 2000). Another example of oral delivery of insulin with Chitosan nanoparticles is provided by Pan et al., (Intl. J. Pharmaceutics, 249:139, 2002). In addition, polyalkylcyanoacrylate nanocapsules have also been reported to be an effective carrier for oral delivery of insulin in diabetic animals (Damge et al., Diabetes, 37:246, 1988). The uptake of particulate materials by gastrointestinal route is documented and lymphatic Peyer's patches are involved (Hussain et al., Adv. Drug Delivery Rev. 50:107, 2001).

Particle size appears to be one of the critical factors affecting absorption efficiency. For example, Jani et al., (J. Pharm. Pharmacol. 42:821, 1990) studied the intestinal absorption of polystyrene particles in rats and demonstrated the relationship between absorption efficiency and particle size. Similarly, the size dependence in intestinal absorption was also observed in poly(lactide-co-glycolide) particles by Desai et al., (Pharm. Res. 13:1838, 1996).

Nanometer scale particles have been proposed for use as carrier particles for biological macromolecules such as proteins and nucleic acids. See U.S. Pat. Nos. 5,178,882; 5,219,577; 5,306,508; 5,334,394; 5,460,830; 5,460,831; 5,462,750; 5,464,634, 6,355,271.

Calcium phosphate particles are bio-adhesive/biocompatible and have been routinely used as carrier to deliver nucleic acid into intracellular compartments in vitro (Chen et al., Mol. Cell. Biol. 7:2745-52, 1987). In addition, calcium phosphate has also been tested as carrier for genetic therapy to delivery large nucleic acid in vivo (Roy et al., Intl. J. Pharmaceutics 250:25, 2003).

Therapeutic calcium phosphate particles have been described. See U.S. Pat. Nos. 6,355,271; 6,183,803; U.S. Pub. Nos. 2005/0234114; 2004/0258763; 2002/0054914; 2002/0068090; 2003/0185892; 2001/0048925; WO 02/064112; WO 03/051394; WO 00/46147; WO 2004/050065. The effect of oral formulation of insulin loaded calcium phosphate particles is tested in diabetic mice and control of blood glucose has been shown (Morcol et al., Intl. J. Pharmaceutics 277:91, 2004). However, the particle size in this study was in the range of 2-4 μm, which is clearly not optimal.

To make calcium phosphate particles with desired size, extensive sonication is required (Cherian et al., Drug Dev. Ind. Pharmacy, 26:459, 2000; Roy et al., Intl. J. Pharmaceutics 250:25, 2003), which may damage delicate macromolecule drugs encapsulated.

Furthermore, the encapsulating efficiency of macromolecules into calcium phosphate particles is often low. For example, U.S. Pat. No. 6,355,271 discloses absorption efficiency of about 40% if insulin is added to preformed calcium phosphate particles; and about 89%, if insulin is mixed during the particle formation.

These reported methods either result in particles with less optimal size, or require harsh conditions such as extended sonication that are not compatible to macromolecule formulation. Therefore, there remains a need for oral macromolecule delivery system that is highly efficient and easily produced with low cost.

### SUMMARY OF THE INVENTION

In one aspect, the invention provides a plurality of particles comprising: a) a plurality of calcium phosphate core nanoparticles; b) a GLP-1 agonist encapsulated in the core nanoparticles; and c) a co-precipitating agent encapsulated in the core nanoparticles to enhance the encapsulation efficiency of the GLP-1 agonist into the core nanoparticles relative to corresponding calcium phosphate core nanoparticles that do not comprise the bile salt. In some embodiments, the GLP-1 agonist is exenatide or a physiologically acceptable salt or derivative thereof. In some embodiments, the co-precipitating agent comprises a bile salt selected from the group consisting of a cholate, a deoxycholate, a taurocholate, a glycocholate, a taurodeoxycholate, an ursodeoxycholate, a tauroursodeoxycholate, a chenodeoxycholate, and a combination thereof.

In another aspect, the invention provides a plurality of particles comprising: a) a plurality of calcium phosphate core nanoparticles; b) a biologically active macromolecule encapsulated in the core nanoparticles; and c) a co-precipitating agent encapsulated in the core nanoparticles to enhance the encapsulation efficiency of the biologically active macromolecule into the core nanoparticles relative to corresponding calcium phosphate core nanoparticles that does not comprise the fatty acid salt. In some embodiments, the co-precipitating agent comprises a fatty acid salt selected from the group consisting of a caproate, a caprylate, a pelargonate, a caprate, a laurate, a myristate, and a combination thereof.

In yet another aspect, the invention provides a method of making calcium phosphate particles, the method comprising: a) contacting an aqueous solution of a calcium salt with an aqueous solution of a phosphate salt in the presence of a co-precipitating agent; b) mixing the resulting solution in step a) until a calcium phosphate particle of a desired size is obtained; and c) recovering the calcium phosphate particles. In some embodiments, the method comprises a further step of adding a biologically active macromolecule into the aqueous solution of the phosphate salt or the aqueous solution of the calcium salt before contacting the aqueous solution of the calcium salt with the aqueous solution of the phosphate salt in the presence of the co-precipitating agent, whereby the calcium phosphate particles are co-crystallized with the biologically active macromolecule. In some embodiments, the co-precipitating agent comprises a fatty acid salt selected from the group consisting of a caproate, a caprylate, a pelargonate, a caprate, a laurate, a myristate, and a combination thereof.

In a further aspect, the invention provides pharmaceutical compositions comprising the calcium phosphate particles of the present invention and a pharmaceutically acceptable carrier. In some embodiments, the pharmaceutical compositions are in the form of capsules, tablets, spheres, or powder. In some embodiments, the pharmaceutical compositions further comprise an enteric coating and/or an absorption enhancer.

In a further aspect, the invention provides a method of treating a subject in need of a biologically active macromolecule treatment, the method comprising administering to the subject a therapeutically effective amount of a pharmaceutical composition comprising the calcium phosphate particles of the present invention. In some embodiments, the pharmaceutical composition is administered via the oral route. In some embodiments, the biologically active macromolecule is a GLP-1 agonist, such as exenatide or a physiologically acceptable salt or derivative thereof

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted.

The discussion of the general methods given herein is intended for illustrative purposes only. Other alternative methods and embodiments will be apparent to those of skill in the art upon review of this disclosure.

As used herein, “a” or “an” means “at least one” or “one or more.”

A group of items linked with the conjunction “or” should not be read as requiring mutual exclusivity among that group, but rather should also be read as “and/or” unless expressly stated otherwise.

As used herein, the terms “treatment” or “treating” refers to any manner in which the symptoms of a condition, disorder or disease are ameliorated or otherwise beneficially altered. In the context of treating a hematological malignancy, the hematological malignancy can be onset, relapsed or refractory. Full eradication of the condition, disorder or disease is not required. Amelioration of symptoms of a particular disorder refers to any lessening of symptoms, whether permanent or temporary, that can be attributed to or associated with administration of a therapeutic composition of the present invention or the corresponding methods and combination therapies. Treatment also encompasses pharmaceutical use of the compositions in accordance with the methods disclosed herein.

As used herein, the term “subject” is not limited to a specific species or sample type. For example, the term “subject” may refer to a patient, and frequently a human patient. However, this term is not limited to humans and thus encompasses a variety of mammalian species.

As used herein, the terms “administration” or “administering” refers to any suitable method of providing a composition of the present invention to a subject. It is not intended that the present invention be limited to any particular mode of administration. In some embodiments, the pharmaceutical compositions of the present invention are administered via the oral route. In other embodiments, the compounds and pharmaceutical compositions of the present invention are administered via a parenteral route, e.g., via intramuscular, intraperitoneal, intravenous, intracisternal or subcutaneous injection or infusion. The pharmaceutical compositions may be formulated in suitable dosage unit formulations appropriate for each route of administration.

As used herein, the term “effective amount” or “therapeutically effective amount” of a compound refers to a nontoxic but sufficient amount of the compound to provide the desired therapeutic or prophylactic effect to most patients or individuals. In the context of treating a hematological malignancy, a nontoxic amount does not necessarily mean that a toxic agent is not used, but rather means the administration of a tolerable and sufficient amount to provide the desired therapeutic or prophylactic effect to a patient or individual. The effective amount of a pharmacologically active compound may vary depending on the route of administration, as well as the age, weight, and sex of the individual to which the drug or pharmacologically active agent is administered. Those of skill in the art given the benefit of the present disclosure can easily determine appropriate effective amounts by taking into account metabolism, bioavailability, and other factors that affect plasma levels of a compound following administration within the unit dose ranges disclosed further herein for different routes of administration.

As used herein, the term “encapsulated,” “embedded” or “incorporated” means complexed, encased, bonded with, related to, coated with, layered with, or enclosed by a substance. Thus, a substance encapsulated in a particle means the substance is incorporated into the particle structure, or coated or attached to the surface of the particle, or both.

As used herein, the term “composition” refers to a product comprising the specified ingredients in the specified amounts, as well as any product which results, directly or indirectly, from combination of the specified ingredients in the specified amounts.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As noted above, the present invention provides a plurality of particles comprising: a) a plurality of calcium phosphate core nanoparticles; b) a GLP-1 agonist encapsulated in the core nanoparticles; and c) a co-precipitating agent encapsulated in the core nanoparticles to enhance the encapsulation efficiency of the GLP-1 agonist into the core nanoparticles relative to corresponding calcium phosphate core nanoparticles that do not comprise the bile salt.

As used herein, the term “GLP-1 agonist” refers to compounds that which fully or partially activate the human GLP-1 receptor. Glucagon-like peptide 1 (GLP-1) is released from the L, cells in the intestine and serves to augment the insulin response after oral intake of glucose or fat. The term includes GLP-1 peptides, as well as variants, analogs, and derivatives thereof. For example, GLP-1 peptides comprise the wild type glucagon-like peptide, truncations, elongations, mutations, or other variations thereof. The term includes analogs such as ZP10A or BIM-51077, a GLP-1 or its analog conjugated to polyethylene glycol, a GLP-1 or its analog fused with albumin such as albugon, or chemically conjugated to the albumin such as liraglutide or CJC-1131. Similarly, extendin-4, also referred to as exenatide, is a GLP-1 agonist, and is included in the term “GLP-1 agonist” along with its analogs and derivatives. Exenatide is disclosed in U.S. Pat. No. 5,424,286 and marketed under the trademark BYETTA®. Accordingly, exenatide, exenatide analogs such as, for example, those disclosed in U.S. Pat. No. 7,329,646, and long-acting conjugates such as CJC-1134, are all contemplated within the present invention.

GLP-1 agonists are useful for treating diabetes, for stimulating insulin release, for treating hyperglycemia, for treating dyslipidemia, for treating and preventing cardiovascular diseases, for reducing morbidity and mortality after stroke, for increasing urine flow, for lowering plasma glucagon, for reducing gastric motility and/or delaying gastric emptying, for treating obesity, Type II diabetes, eating disorders, and insulin-resistance syndrome. The methods are also useful for lowering the plasma glucose level, lowering the plasma lipid level, reducing the cardiac risk, reducing the appetite, and reducing the weight of subjects. When lowering plasma glucagon, such methods may be employed to treat hyperglucagonemia, such as in patients with necrolytic migratory erythema, patients with a glucagonomas, patients with a diabetes-related disorder, such as but not limited to Type II diabetes. With respect to treating cardiovascular diseases, GLP-1 agonists are useful for treating myocardial infarct, acute coronary syndrome (ACS), unstable angina (UA), non-Q-wave cardiac necrosis (NQCN), left ventricular hypertrophy, coronary artery disease, essential hypertension, acute hypertensive emergency, cardiomyopathy, heart insufficiency, exercise tolerance, chronic heart failure, arrhythmia, cardiac dysrhythmia, syncopy, atheroschlerosis, mild chronic heart failure, angina pectoris, cardiac bypass reocclusion, intermittent claudication (atheroschlerosis oblitterens), diastolic dysfunction and systolic dysfunction.

As noted above, the compositions described herein provide for an increase in the encapsulation efficiency of the biologically active macromolecules relative to a calcium phosphate nanoparticle that does not comprise the co-precipitating agent. The encapsulation efficiency in the presence of the co-precipitating agent may be greater than or equal to 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%.

The small size of the present particles is an important factor that significantly influences the particles' effectiveness as a drug delivery vehicle. Accordingly, in some embodiments, the average diameter of the core nanoparticles is less than about 1000 nm, preferably less than about 300 nm, less than about 200 nm, less than about 100 nm, or less than about 50 nm.

In some embodiments, the co-precipitating agent may comprise a chemical in which the calcium salt has low aqueous solubility, and which can substantially absorb a biologically active macromolecule. In some embodiments, the co-precipitating agent may comprise a bile salt; which include conjugated or un-conjugated bile acids, examples include, but are not limited to, a cholate, a deoxycholate, a taurocholate, a glycocholate, a taurodeoxycholate, an ursodeoxycholate, a tauroursodeoxycholate, a chenodeoxycholate, as well as derivatives and combinations thereof. In other embodiments, the co-precipitating agent may comprise a fatty acid salt. Examples include, but are not limited to, a caproate, a caprylate, a pelargonate, a caprate, a laurate, a myristate, a palmitate, a stearate, an arachidate, as wells as derivatives and combinations thereof.

In some embodiments, the particles are adapted to deliver the biologically active macromolecule to a mucosal surface. In some embodiments, the particles are adapted to deliver the biologically active macromolecule via the oral route to a subject in need thereof.

In some embodiments, the particles may further comprise a bioadhesive coating, which enhances their adherence to the mucosal membrane. The bioadhesive coating may comprise such materials as carbomer, polycarbophil, chitosan, alginate, thiomer, gelatin, hydroxyl propyl methyl cellulose, carboxymethyl cellulose, polyvinyl alcohol, polyethylene glycol, polyvinyl pyrrolidone, fumaric anhydride oligomer, polyesters, polyacrylates, polysaccharides, modified dextrans, pectin, xanthan gum, as well as salts, derivatives and combinations thereof.

In some embodiments, the particles may further comprise an enteric coating, which comprises pH sensitive polymers that allow the particles to selectively adhere to certain region of the gastrointestinal tract. The enteric coating materials include but not limited to cellulose acetate phthalate, hydroxyl propyl methyl cellulose phthalate, polyvinyl acetate phthalate, various EUDRAGIT® polymers, as well as their salts and their derivatives.

In some embodiments, the particles may further comprise a site selective coating, which comprises polymers that allow the particles to selectively adhere to certain region of the gastrointestinal tract. For example, the coating may be applied to the particles for colon specific delivery and the coating materials includes but are not limited to azo polymers, colon degradable polysaccharides such as pectin, amylose, guar gum, xylan, cyclodextrin, dextran, their salts, derivatives, and combinations thereof.

The invention further encompasses a plurality of particles comprising: a) a plurality of calcium phosphate core nanoparticles; b) a biologically active macromolecule encapsulated in the core nanoparticles; and c) a co-precipitating agent encapsulated in the core nanoparticles to enhance the encapsulation efficiency of the biologically active macromolecule into the core nanoparticles relative to corresponding calcium phosphate core nanoparticles that does not comprise the fatty acid salt.

In some embodiments, the biologically active macromolecule is selected from the group consisting of a protein, a peptide, a polysaccharide, a nucleic acid, a lipid, a carbohydrate, and a combination thereof.

In some embodiments, the protein is selected from the group consisting of an anti-thrombin, an albumin, an alpha-1-proteinase inhibitor, an antihemophilic factor, a coagulation factor, an antibody, an anti-CD20 antibody, an anti-CD52 antibody, an anti-CD33 immunotoxin, a DNase, an erythropoietin, a factor IX, a factor VII, a factor VIII, a follicle stimulating hormone, a granulocyte colony-stimulating factor (G-CSF), a pegylated G-CSF, a galactosidase alpha or beta, a glucagon, a glucocerebrosidase, a granulocyte-macrophage colony-stimulating factor (GM-CSF), a choriogonadotropin, a hepatitis B antigen, a hepatitis B surface antigen, a hepatitis B core antigen, a hepatitis B envelopment antigen, a hepatitis C antigen, a hirudin, an anti-HER-2 antibody, an anti-IgE antibody, an anti-IL-2 receptor antibody, an insulin, an insulin glargine, an insulin aspart, an insulin lispro, an interferon, a pegylated interferon, an interferon alpha or alpha 2a or alpha 2b or concensus, an interferon beta or beta-1a or beta-1b or betaser, an interferon gamma, a interleukin-2, a interleukin-11, a interleukin-12, a luteinizing hormone, a nesiritide, an osteogenic protein-1, an osteogeneic protein-2, a lyme vaccine, a platelet derived growth factor, an anti-platelet antibody, an anti-RSV antibody, a somatotropin, an anti-tumor necrosis factor (TNF) antibody, a TNF receptor-Fc fusion protein, a tissue plasminogen activator (tPA), a TNK-tPA, a thyroid stimulating hormone (TSH), a fibrinolytic enzyme, a thrombolytic enzyme, an adenosine deaminase, a pegylated adenosine deaminase, an anistreplase, an asparaginase, a collagenase, a streptokinase, a sucrase, a urokinase, an aprotinin, a botulinum toxin, a fibroblast growth factor, a vascular endothelia growth factor, and a venom. The proteins may be produced by recombinant technology, chemical synthesis or extracted from biological sources. The proteins also include the mutants and modified analogs or derivatives. The origin of the proteins may be from human or other species.

In some embodiments, the peptide is selected from the group consisting of an

ACTH, an anti-angiogenic peptide, an adamtsostatin, an adiponectin, an adipokinetic hormone, an deiponutrin, an adipose desnutrin, an adrenomedullin, an agouti-related protein, an alarin, an allatostatin, an amelogenin, a calcitonin, an amylin, an amyloid, an agiopoietin, an angiotensin, an anorexigenic peptide, an anti-inflammatory peptide, an anti-diuretic factor, an anti-microbial peptide, an apelin, an apidaecin, a RGD peptide, an atrial natriuretic peptide, an atriopeptin, an auriculin, an autotaxin, a bombesin, a bombinakinin, a bradykinin, a brain natriuretic peptide, a brain-derived neutrophic factor, a brevinin, a C-peptide, a caspase inhibitor, a pancreatic peptide, a buccalin, a bursin, a C-type natriuretic peptide, a calcitonin related peptide, a calcitonin receptor stimulating peptide, a calmodulin, a CART, a cartilostatin, a casomokinin, a casomorphin, a catestatin, a cathep sin, a cecropin, a cerebellin, a chemerin, a chelocystokinin, a chromogranin, a ciliary neutrophic factor, a conantokin, a conopressin, a conotoxin, a copeptin, a cortical androgen stimulating hormone, a corticotropin release factor, a cortistatin, a coupling factor, a defensin, a delta sleep inducing peptide, a dermorphin, a vasopressin, a desamino-vasopressin, a diuretic hormone, a dynorphin, an endokinin, an endomorphin, an endorphin, an endostatin, an endothelin, an enkephalin, an enterostatin, an exendin, an exendin-4, an erythropoietic peptide, an epithelia growth factor, a fat targeted peptide, a galanin, a gastric inhibitory peptide, a gastrin, a gastrin releasing peptide, a ghrelin, a glucagon, a glucagon-like peptide, a glutathione derivative, a gluten exorphin, a growth hormone releasing factor, a GM-CSF inhibitory peptide, a growth hormone peptide, a guanylin, a HIV peptide, a helodemine, a hemokinin, a HCV peptide, a HBV peptide, a HSV peptide, a Herpes virus peptide, a hirudin, a hydra peptide, an insulin-like growth factor, a hydrin, an intermedin, a kassinin, a keratinocyte growth factor, a kinetensin, a kininogen, a kisspeptin, a kyotorphin, a laminin peptide, a leptin peptide, a leucokinin, a leucopyrokinin, a leupeptin, a luteinizing hormone releasing hormone (LHRH), a lymphokine, a melanin concentrating hormone and its inhibitor, a melanocyte stimulating hormone releasing inhibitor, a melanotropin-potentiating factor, a morphine modulating neuropeptide, a MSH, a neoendorphin, a nesfatin, a neurokinin, a neuromedin, a neutropeptide Y, a neurotensin, a neutrotrophic factor, a nociceptin, an obestatin, an opioid receptor antagonist, an orexin, an osteocalcin, an oxytocin, a pancreastatin, a peptide YY, a physalaemin-like peptide, a secretin, a somatostatin, a sperm-activating peptide, a substance P, a syndyphalin, a thrombospondin, a thymopoietin, a thymosin, a thyrotropin-releasing hormone, a transforming growth factor, a tuftsin, a tumor necrosis factor antagonist or related peptide, a usrechistachykinin, a urocortin, a urotensin antagonist, a valorphin, a vasotocin, a VIP, a, xenopsin or related peptide. The peptides may be produced by recombinant technology, chemical synthesis or extracted from biological sources. The peptides include the mutants or modified analogs or derivatives. The origin of the peptides may be from human or other species.

In some embodiments, the biologically active macromolecule is a vaccine selected from the group consisting of a adenovirus, anthrax, BCG, botulinum, cholera, diphtheria toxoid, diphtheria & tetanus toxoids, diphtheria tetanus & pertussis, haemophilus B, hepatitis A, hepatitis B, influenza, encephalitis, measles, mumps, rubella, meningococcal, plague, pertussis, pneumococcal, polio, rabies, rotavirus, rubella, smallpox, tetanus toxoid, typhoid, varicella, yellow fever, bacterial antigens and any combination thereof.

In some embodiments, the biologically active macromolecule is an allergen selected from the group consisting of house dust mice, animal dander, molds, pollens, ragweed, latex, vespid venoms and insect-derived allergens, and any combinations thereof.

In some embodiments, the biologically active macromolecule is selected from the group consisting of a GLP-1 agonist, an insulin, an erythropoietin, an interferon, a growth hormone, a PTH, a calcitonin, a leuprolide, and a derivative thereof. In some embodiments, the GLP-1 agonist is exenatide or physiologically acceptable salts or derivatives thereof.

In some embodiments, the biologically active macromolecules listed above may comprise a family of related molecules, including the wild type molecule with original structure, analogs with modified structure or sequence, and chemically or biologically modified analogs.

The invention further encompasses pharmaceutical compositions comprising the calcium phosphate particles of the present invention and a pharmaceutically acceptable carrier.

Suitable carriers and their formulations are known in the art and described in Remington, The Science and Practice of Pharmacy 20th Ed. Mack Publishing, 2000. The pharmaceutical composition may be formulated in the form of solution, capsule, tablet, powder, and aerosol; and may be formulated in the form suitable for oral delivery, mucosal delivery, or delivery to an ocular surface. The composition may include other components, such as buffers, preservatives, nonionic surfactants, solubilizing agents, absorption enhancers, stabilizing agents, emollients, lubricants and tonicity agents. The composition may be formulated to achieve controlled release for the macromolecules.

In some embodiments, the particles are formulated in a pharmaceutical acceptable carrier in the form of capsules, tablets, particles, liquids, gels, pastes, and/or creams. The pharmaceutical compositions may be administered by any suitable means, for example, orally, such as in the form of tablets, capsules, granules or powders; sublingually; buccally; parenterally, such as by subcutaneous, intravenous, intramuscular, intra(trans)dermal, or intracisternal injection or infusion techniques (e.g., as sterile injectable aqueous or non-aqueous solutions or suspensions); nasally such as by inhalation spray or insufflation; topically, such as in the form of a cream or ointment ocularly in the form of a solution or suspension; vaginally in the form of pessaries, tampons or creams; or rectally such as in the form of suppositories; in dosage unit formulations containing non-toxic, pharmaceutically acceptable vehicles or diluents. The nanoparticles may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps.

The pharmaceutical compositions for the administration of the compounds of the invention may conveniently be presented in dosage unit form and may be prepared by any of the methods well known in the art of pharmacy. These methods generally include the step of bringing the non-particles into association with the carrier which constitutes one or more accessory ingredients. In general, the pharmaceutical compositions are prepared by uniformly and intimately bringing the nanoparticle into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the nanoparticles may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents such as sweetening agents, flavoring agents, coloring agents and preserving agents, e.g. to provide pharmaceutically stable and palatable preparations. Tablets contain the nanoparticles in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, lubricating agents, for example magnesium stearate, stearic acid or talc; and absorption enhancing agents that perturb the lipid bilayer membrane and assist the trans-intestinal absorption of the drug, for example, detergents or surface modulating agents, including EDTA, bile salts, and medium chain fatty acid salts, such as caproate, caprylate, pelargonate, caprate, laurate, and myristate. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release.

Aqueous suspensions contain the nanoparticles in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the nanoparticles in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the nanoparticles in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectable formulations.

For administration to the respiratory tract, including intranasal administration, the active compound may be administered by any of the methods and formulations employed in the art for administration to the respiratory tract.

Thus in general the nanoparticles may be administered in the form of a solution or a suspension or as a dry powder.

Solutions and suspensions will generally be aqueous, for example prepared from water alone (for example sterile or pyrogen-free water) or water and a physiologically acceptable co-solvent (for example ethanol, propylene glycol or polyethylene glycols such as PEG 400).

Such solutions or suspensions may additionally contain other excipients for example preservatives (such as benzalkonium chloride), solubilising agents/surfactants such as polysorbates (e.g., Tween 80, Span 80, benzalkonium chloride), buffering agents, isotonicity-adjusting agents (for example sodium chloride), absorption enhancers and viscosity enhancers. Suspensions may additionally contain suspending agents (for example microcrystalline cellulose and carboxymethyl cellulose sodium).

Solutions or suspensions are applied directly to the nasal cavity by conventional means, for example with a dropper, pipette or spray. The formulations may be provided in single or multidose form. In the latter case a means of dose metering is desirably provided. In the case of a dropper or pipette this may be achieved by the subject administering an appropriate, predetermined volume of the solution or suspension. In the case of a spray this may be achieved for example by means of a metering atomising spray pump.

Administration to the respiratory tract may also be achieved by means of an aerosol formulation in which the compound is provided in a pressurised pack with a suitable propellant, such as a chlorofluorocarbon (CFC), for example dichlorodifluoromethane, trichlorofluoromethane or dichlorotetrafluoroethane, carbon dioxide or other suitable gas. The aerosol may conveniently also contain a surfactant such as lecithin. The dose of active compound may be controlled by provision of a metered valve.

Alternatively the active compound may be provided in the form of a dry powder, for example a powder mix of the nanoparticles in a suitable powder base such as lactose, starch, starch derivatives such as hydroxypropylmethyl cellulose and polyvinylpyrrolidine (PVP). Conveniently the powder carrier will form a gel in the nasal cavity. The powder composition may be presented in unit dose form, for example in capsules or cartridges of e.g., gelatin, or blister packs from which the powder may be administered by means of an inhaler.

In formulations intended for administration to the respiratory tract, including intranasal formulations, the nanoparticles will generally have a small particle size, for example of the order of 5 microns or less. Such a particle size may be obtained by means known in the art if necessary, for example, by micronisation.

When desired, formulations adapted to give sustained release of the active compound may be employed.

The nanoparticles of the present invention may also be administered in the form of suppositories for rectal administration of the drug. These compositions can be prepared by mixing the nanoparticles with a suitable non-irritating excipient which is solid at ordinary temperatures but liquid at the rectal temperature and will therefore melt in the rectum to release the drug. Such materials are cocoa butter and polyethylene glycols.

Compositions suitable for vaginal administration may be presented as pessaries, tampons, creams, gels, pastes, foams or sprays containing in addition to the active ingredient such carriers as are known in the art to be appropriate.

For topical use, creams, ointments, jellies, solutions or suspensions, etc., containing the nanoparticles of the present invention are employed. (For purposes of this application, topical application shall include mouthwashes and gargles.)

The nanoparticles may also be presented for use in the form of veterinary compositions, which may be prepared, for example, by methods that are conventional in the art. Examples of such veterinary compositions include those adapted for: (a) oral administration, external application, for example drenches (e.g. aqueous or non-aqueous solutions or suspensions); tablets or boluses; powders, granules or pellets for admixture with feed stuffs; pastes for application to the tongue; (b) parenteral administration for example by subcutaneous, intramuscular or intravenous injection, e.g. as a sterile solution or suspension; or (when appropriate) by intramammary injection where a suspension or solution is introduced in the udder via the teat; (c) topical applications, e.g. as a cream, ointment or spray applied to the skin; or (d) rectally or intravaginally, e.g. as a pessary, cream or foam.

The invention further encompasses a method of making calcium phosphate particles, the method comprising: a) contacting an aqueous solution of a calcium salt with an aqueous solution of a phosphate salt in the presence of a co-precipitating agent; b) mixing the resulting solution in step a) until a calcium phosphate particle of a desired size is obtained; and c) recovering the calcium phosphate particles.

In some embodiments, the concentration of the calcium salt in the aqueous solution ranges from about 5 mM to about 200 mM. In some embodiments, the concentration of the phosphate salt in the aqueous solution is ranges from about 5 mM to about 200 mM.

In some embodiments, the method further comprises the step of adding a biologically active macromolecule into the aqueous solution of the phosphate salt or the aqueous solution of the calcium salt before contacting the aqueous solution of the calcium salt with the aqueous solution of the phosphate salt in the presence of the co-precipitating agent, whereby the calcium phosphate particles are co-crystallized with the biologically active macromolecule.

As described herein, the co-precipitating agent may comprise a bile salt, a fatty acid salt, or a combination thereof. In some embodiments, the bile salt may be selected from the group consisting of a cholate, a deoxycholate, a taurocholate, a glycocholate, a taurodeoxycholate, an ursodeoxycholate, a tauroursodeoxycholate, a chenodeoxycholate, and a combination thereof. In some embodiments, the fatty acid salt may be selected from the group consisting of a caproate, a caprylate, a pelargonate, a caprate, a laurate, a myristate, and a combination thereof.

In some embodiments, the concentration of the co-precipitating agent ranges from about 0.01% to about 5%, from about 0.2% to about 3%, or from about 0.5% to about 1.5%.

The particles of the invention may be further coated or impregnated, or both with surface modifying agents. Such surface modifying agents suitable for use in the present invention include substances that facilitate the binding or entrapment of biologically active macromolecules to the particle, without denaturing the macromolecule. Examples of suitable surface modifying agents are described in U.S. Pat. Nos. 5,460,830, 5,462,751, 5,460,831, and 5,219,577. Other examples of suitable surface modifying agents may include basic or modified sugars, such as cellobiose, or oligonucleotides described in U.S. Pat. No. 5,219,577. Suitable surface modifying agents also include carbohydrates, carbohydrate derivatives, and other macromolecules with carbohydrate-like components characterized by the abundance of —OH side groups, as described, for example, in U.S. Pat. No. 5,460,830. Polyethylene glycol (PEG) is a particularly suitable surface modifying agent.

Coating of calcium phosphate particles may be prepared by adding a stock solution of a surface modifying agent, such as cellobiose or PEG (e.g., around 292 mM) to a suspension of calcium phosphate core particles at a ratio of about 1 ml of stock solution to about 20 ml of particle suspension. The mixture can be swirled and allowed to stand overnight to form at least partially coated core particles. Generally, this procedure will result in substantially complete coating of the particles, although some partially coated or uncoated particles may be present.

In a further aspect, the invention provides a method of treating a subject in need of a biologically active macromolecule treatment, the method comprising administering to the subject a therapeutically effective amount of a pharmaceutical composition comprising the calcium phosphate particles of the present invention. In one preferred embodiment, the biologically active macromolecule is a GLP-1 agonist, such as exenatide or a physiologically acceptable salt or derivative thereof

Administration of the composition of the invention may be by any means known in the art, including: orally, intravenously, subcutaneously, via inhalation, intraarterially, intramuscularly, intracardially, intraventricularly, parenteral, intrathecally, and intraperitoneally. Administration may be systemic, e.g. intravenously, or localized. In some embodiments, the pharmaceutical composition is administered to a mucosal surface. In some embodiments, the pharmaceutical composition is administered via the oral route.

The following examples are included for illustrative purposes and are not intended to limit the scope of the invention.

### EXAMPLES

### Example 1

### Effect of Ursodeoxycholate on Encapsulation Efficiency of Exenatide into Calcium Phosphate Nanoparticles

To evaluate the effect of bile salts on the encapsulation efficiency of exenatide into calcium phosphate nanoparticles, take two 50 ml centrifuge tubes and add components as listed in the table below. Two hundred milligram polyethylene glycol (PEG, MW6000), either 0 or 70 mg deoxycholate (dissolved into ethanol and neutralized with equal molar NaOH), 20 mM HEPES buffer pH 6.9, 0.4 ml 2.5 M Na₂HPO₄, were added and the final volume was adjusted to 10 ml with distilled water. The solutions were labeled A1 and A2, and their compositions are summarized in Table 1.

TABLE 1

| | A1 | A2 |
|---|---|---|
| PEG (%) | 1 | 1 |
| UDCA (%) | 0 | 0.7 |
| Phosphate (mM) | 20 | 20 |
| Volume (ml) | 10 | 10 |

In two separate 50 ml centrifuge tubes, 60 mM CaCl₂ and 1.5 mg/ml exenatide were added in 10 ml solutions and labeled B1 and B2. Optic density at 280 nm was measured for both B solutions. Calcium phosphate nanoparticles were formed by slowly mixing corresponding A and B solutions. Precipitation was seen immediately and the gentle mixing was continued at room temperature for 5 min. The mixture was spun down at 8000 rpm for 10 min. Optical density at 280 nm of the supernatant was measured and the encapsulation efficiency was calculated using the following equation:

Efficiency (%)=[1-(OD280 of supernatant×20)/(OD280 of solution B×10)]×100%

The encapsulation efficiencies of the resulting nanoparticles are summarized in Table 2.

TABLE 2

| | 1 | 2 |
|---|---|---|
| UDCA (%) | 0 | 0.35 |
| Efficiency (%) | 9.3 | 92.9 |

The result shows that exenatide is poorly encapsulated into calcium phosphate nanoparticles in the absence of UDCA, and the presence of UDCA significantly enhances (10-fold) the encapsulation efficiency of exenatide.

### Example 2

### Effect of Caprate on Encapsulation Efficiency of Exenatide into Calcium Phosphate Nanoparticles

To evaluate the effect of caprate on the encapsulation efficiency of exenatide into calcium phosphate nanoparticles, take three 50 ml centrifuge tubes and add components as listed in the table below. Two hundred milligram polyethylene glycol (PEG, MW 6000), either 0, 50 or 100 mg sodium caprate dissolved in ethanol, 20 mM HEPES buffer pH 6.9, and 20 mM Na₂HPO₄, were added and final volume was adjusted to 10 ml with distilled water. The solutions were labeled A1-A3, and their compositions are summarized in Table 3.

TABLE 3

| | A1 | A2 | A3 |
|---|---|---|---|
| PEG (%) | 1 | 1 | 1 |
| Caprate (%) | 0 | 0.5 | 1.0 |
| Phosphate (mM) | 20 | 20 | 20 |
| Volume (ml) | 10 | 10 | 10 |

In three separate 50 ml centrifuge tubes, 60 mM CaCl₂ and 1.5 mg/ml exenatide were added in 10 ml solutions and labeled B1-B3. Optic density at 280 nm was measured for all B solutions. Calcium phosphate nanoparticles were formed by slowly mixing corresponding A and B solutions. Precipitation was seen immediately and the gentle mixing was continued at room temperature for 5 min. The mixture was spun down at 8000 rpm for 10 min. Optical density at 280 nm of the supernatant was measured and the encapsulation efficiency was calculated as in Example 1. The encapsulation efficiencies of the resulting nanoparticles are summarized in Table 4.

TABLE 4

| | 1 | 2 | 3 |
|---|---|---|---|
| Caprate (%) | 0 | 0.25 | 0.5 |
| Efficiency (%) | 9.3 | 40.7 | 61.1 |

The result shows that exenatide is poorly encapsulated into calcium phosphate nanoparticles in the absence of caprate, and the presence of caprate significantly enhances the encapsulation efficiency of exenatide in a dose dependent fashion.

### Example 3

### Effect of Caprate on Encapsulation Efficiency of Insulin into Calcium Phosphate Nanoparticles

To evaluate the effect of caprate on the encapsulation efficiency of insulin into calcium phosphate nanoparticles, take three 50 ml centrifuge tubes and add components as listed in the table below. Two hundred milligram polyethylene glycol (PEG, MW 6000), either 0, 39 or 117 mg sodium caprate, 20 mM HEPES buffer pH 6.9, and 20 mM Na₂HPO₄, were added and final volume was adjusted to 10 ml with distilled water. The solutions were labeled A1-A3, and their compositions are summarized in Table 5.

TABLE 5

| | A1 | A2 | A3 |
|---|---|---|---|
| PEG (%) | 1 | 1 | 1 |
| Caprate (%) | 0 | 0.39 | 1.17 |
| Phosphate (mM) | 10 | 10 | 10 |
| Volume (ml) | 10 | 10 | 10 |

In three separate 50 ml centrifuge tubes, 60 mM CaCl₂ and 1 mg/ml insulin were added in 10 ml solutions and labeled B1-B3. Optical density at 280 nm was measured for all B solutions. Calcium phosphate nanoparticles were formed by slowly mixing corresponding A and B solutions. Precipitation was seen immediately and the gentle mixing was continued at room temperature for 5 min. The mixture was spun down at 8000 rpm for 10 min. Optical density at 280 nm of the supernatant was measured and the encapsulation efficiency was calculated as in Example 1. The encapsulation efficiencies for the resulting nanoparticles are summarized in Table 6.

TABLE 6

| | 1 | 2 | 3 |
|---|---|---|---|
| Caprate (%) | 0 | 0.2 | 0.6 |
| Efficiency (%) | 0.2 | 73.8 | 85.3 |

The result shows that in the presence of caprate, the encapsulation efficiency of insulin into calcium phosphate nanoparticles was significantly improved in a dose dependent fashion.

### Example 4

### In vivo Activity of Exenatide Encapsulated Calcium Phosphate Nanoparticles

The activity of exenatide encapsulated calcium phosphate nanoparticles was evaluated in the oblob diabetic mice model.

### Animals.

Thirty-two 8-10 week-old oblob mice (Jackson Labs) were caged in a ventilated room. Food and water were supplied ad librium. Light cycle was set for every 12 hours.

### Treatment.

Mice were fasted for 2 hours, fasting blood glucose was measured, and mice were randomly divided into four groups with 8 mice in each group:
a. Blank, treated with one blank capsule,
b. SC, BYETTA® subcutaneous injection, 0.5 μg/mouse,
c. PO1, Oral exenatide, one capsule or 1 μg formulated exenatide/mouse, formulated according to Example 1, and
d. PO5, Oral exenatide, one capsule or 5 μg formulated exenatide/mouse, formulated according to Example 1.

Capsules were administered by a gavage needle, and BYETTA® was administered subcutaneously. Blood samples were drawn from the tail vein at 0, 0.5, 1, 2, 4, 6, 8, and 10 hours, and the blood glucose level was determined with a glucometer (ReliOn Ultima). Two animals died after capsule administration and were excluded from the analysis.

The effect of oral exenatide on the fasting blood glucose is shown in Table 7. With 1 μg oral exenatide, the blood glucose level decreased compared with blank control but the level did not reach statistical significance. With 5 μg oral exenatide, the reduction of blood glucose level achieved statistical significance at most of the time points.

TABLE 7
The effect of exenatide treatment on blood glucose in mice

| Group | n | 0.5 hr | 1 hr | 2 hr | 4 hr | 6 hr | 8 hr | 10 hr |
|---|---|---|---|---|---|---|---|---|
| Blank | 7 | 153 ± 40 | 141 ± 26 | 138 ± 32 | 91 ± 31 | 100 ± 22 | 103 ± 33 | 124 ± 34 |
| SC | 8 | 80 ± 23\hat{ } | 53 ± 13\hat{ } | 37 ± 9\hat{ } | 37 ± 11\hat{ } | 46 ± 17\hat{ } | 54 ± 24\hat{ } | 65 ± 20\hat{ } |
| PO1 | 7 | 134 ± 24 | 124 ± 44 | 106 ± 33 | 86 ± 17 | 107 ± 59 | 107 ± 39 | 117 ± 44 |
| PO5 | 8 | 104 ± 32* | 107 ± 56 | 65 ± 28* | 53 ± 20* | 69 ± 24* | 82 ± 26 | 89 ± 20* |

\hat{ }p < 0.005;
*p < 0.05

The results indicate that orally delivered exenatide significantly decreased the fasting blood glucose level in a dose dependent fashion.

### Example 5

### Reduction of Fasting Blood Glucose by Oral Exenatide in Diabetic Patients

The feasibility of the oral exenatide encapsulated in calcium phosphate nanoparticles was evaluated in diabetic patients. Nineteen type II diabetic patients volunteered for the study. The study design was open label, randomized, and cross-over. Three cycles of evaluation were performed for each patient.

In the first cycle, each patient was fasted for more than 12 hours. The blood glucose levels were monitored in the next morning. A ReliOn Ultima was used to determine the blood glucose level with finger prick sampling. No treatment was performed in this cycle to establish the baseline.

In the second cycle, each patient was fasted for more than 12 hours. The blood glucose levels were measured the next morning. Each patient was given 5 μg of BYETTA® by subcutaneous injection at time 0 and the fasting blood glucose level was monitored for 6 hours after treatment.

In the third cycle, each patient fasted for more than 12 hours and the fasting blood glucose levels were measured the next morning. Each patient was given either 25, 50, 75 or 100 μg oral exenatide formulation formulated as described in Example 1, and the fasting blood glucose level was monitored for 6 hours after treatment. Each patient was given oral exenatide once.

The sequence of measurement for each patient was arbitrary and there was a 2 day wash out period after each cycle. After the study, there were 18 patient data in the baseline cycle, 18 patient data in BYETTA® injection cycle, 5 patient data in 25 and 50 μg oral exenatide treatment, and 4 patient data in the 75 and 100 μg oral exenatide treatment.

The percent blood glucose change compared with the glucose levels before treatment in each cycle is shown in Table 8. Subcutaneous injection of BYETTA® produced significant reduction of blood glucose compared with baseline cycle. Each of the oral exenatide treatments showed slower reduction of blood glucose at 2 hour after treatment and similar level of blood glucose reduction at 6 hour after treatment compared with subcutaneous injection of BYETTA®.

TABLE 8
The effect of exenatide treatment on blood
glucose in diabetic human patients

| Cycle | n | 1 hr | 2 hr | 4 hr | 6 hr |
|---|---|---|---|---|---|
| Blank | 18 | 9 ± 11 | 6 ± 10 | −6 ± 18 | −13 ± 16 |
| SC | 18 | 0 ± 14 | −20 ± 18 | −24 ± 17 | −26 ± 13 |
| PO25 | 5 | 3 ± 5 | 1 ± 13 | −20 ± 2 | −22 ± 6 |
| PO50 | 5 | 5 ± 12 | −6 ± 13 | −18 ± 14 | −21 ± 9 |
| PO75 | 4 | 2 ± 6 | −8 ± 11 | −19 ± 8 | −22 ± 4 |
| PO100 | 4 | 7 ± 8 | 5 ± 6 | −13 ± 8 | −24 ± 9 |

This study demonstrates that oral administration of exenatide encapsulated in calcium phosphate nanoparticles can achieve a significant reduction of blood glucose levels in diabetic patients.

### Example 6

### Effect of Insulin Concentration on Encapsulation Efficiency in the Presence of UDCA

To evaluate the effect of insulin concentration on the encapsulation efficiency into calcium phosphate nanoparticles, a solution containing 20 mg/ml polyethylene glycol (PEG, MW 10000, Fluka), 20 mM HEPES, pH 6.964, 7.5 mg/ml sodium ursodeoxycholate, 10 mM Na₂HPO₄ was prepared. A second solution containing 60 mM CaCl₂ with either 1 or 4 mg/ml insulin was also prepared. The final volume of both solutions was adjusted to 20 ml and an aliquot was taken from sample containing insulin to determine the optical density at 280 nm. Under stiffing, the two solutions were mixed and precipitation was seen immediately. Stirring was continued at room temperature for 5 min and solutions were centrifuged at 10000 rpm for 15 min. Optical densities of the supernatants at 280 nm were measured to estimate the encapsulation efficiencies as in Example 1. The final concentrations of each component and the resulting encapsulation efficiencies are listed in Table 9.

TABLE 9

| Component | A | B |
|---|---|---|
| PEG (%) | 1 | 1 |
| UDCA (%) | 0.375 | 0.375 |
| HEPES (mM) | 10 | 10 |
| Calcium (mM) | 30 | 30 |
| Phosphate (mM) | 5 | 5 |
| Insulin (mg/ml) | 0.5 | 2 |
| Efficiency (%) | 93.8 | 93.3 |

The result clearly demonstrates that the encapsulation efficiency of insulin in calcium phosphate nanoparticles in the presence of ursodeoxycholate (UDCA) was high regardless of insulin concentration.

### Example 7

### Effect of Insulin Concentration on Encapsulation Efficiency in the Presence of Sodium Caprate

To evaluate the effect of insulin concentration on the encapsulation efficiency into calcium phosphate nanoparticles, a solution containing 20 mg/ml polyethylene glycol (PEG, MW 10000, Fluka), 20 mM HEPES, pH 6.964, 11.7 mg/ml sodium caprate, 10 mM Na₂HPO₄ was prepared. Five calcium chloride solutions containing 60 mM CaCl₂ with 0.2, 0.5, 1, 2 or 4 mg/ml insulin were prepared. The final volume of each solution was adjusted to 20 ml and an aliquot was taken from each insulin-containing sample to determine the optical density at 280 nm. Under stirring, the two solutions were mixed and precipitation was seen immediately. Stirring was continued at room temperature for 5 min and solutions were centrifuged at 10000 rpm for 15 min. Optical densities of the supernatants at 280 nm were measured to estimate the encapsulation efficiencies as in Example 1. The final concentrations of each component and corresponding encapsulation efficiencies are summarized in Table 10.

TABLE 10

| Sample | 1 | 2 | 3 | 4 | 5 |
|---|---|---|---|---|---|
| PEG (%) | 1 | 1 | 1 | 1 | 1 |
| Sodium Caprate (%) | 0.585 | 0.585 | 0.585 | 0.585 | 0.585 |
| HEPES (mM) | 10 | 10 | 10 | 10 | 10 |
| Calcium (mM) | 30 | 30 | 30 | 30 | 30 |
| Phosphate (mM) | 5 | 5 | 5 | 5 | 5 |
| Insulin (mg/ml) | 0.1 | 0.25 | 0.5 | 1 | 2 |
| Efficiency (%) | 77.8 | 88.9 | 87.6 | 73.2 | 58.5 |

This result demonstrates that insulin encapsulation in calcium phosphate nanoparticles by sodium caprate is dependent on insulin concentration, with the optimal insulin concentration falling between 0.25 and 0.5 mg/ml. Encapsulation efficiencies obtained at lower or higher insulin concentrations (0.1 or >1.0 mg/ml, respectively) were lower than those obtained at 0.25 or 0.5 mg/ml insulin. This finding is surprising in view of the observation in Example 6 that the encapsulation efficiency of insulin in the presence of ursodeoxycholate (UDCA) was independent of insulin concentration.

### Example 8

### Effect of Exenatide Concentration on Encapsulation Efficiency in the Presence of Sodium Caprate

To evaluate the effect of exenatide concentration on the encapsulation efficiency into calcium phosphate nanoparticles, a solution containing 20 mg/ml polyethylene glycol (PEG, MW 10000, Fluka), 20 mM HEPES, pH 6.964, 11.7 mg/ml sodium caprate, 10 mM Na₂HPO₄ was prepared. Five calcium chloride solutions containing 60 mM CaCl₂ with 0.2, 0.5, 1, 2 or 4 mg/ml exenatide were prepared. The final volume of each solution was adjusted to 20 ml and an aliquot was taken from each exenatide-containing sample to determine the optical density at 280 nm. Under stirring, the two solutions were mixed and precipitation was seen immediately. Stirring was continued at room temperature for 5 min and solutions were centrifuged at 10000 rpm for 15 min. The final concentration of each component is listed in the table below. Optical densities of the supernatants at 280 nm were measured to estimate the encapsulation efficiencies as in Example 1. The final concentrations of each component and corresponding encapsulation efficiencies are summarized in Table 11.

TABLE 11

| Sample | 1 | 2 | 3 | 4 | 5 |
|---|---|---|---|---|---|
| PEG (%) | 1 | 1 | 1 | 1 | 1 |
| Sodium Caprate (%) | 0.585 | 0.585 | 0.585 | 0.585 | 0.585 |
| HEPES (mM) | 10 | 10 | 10 | 10 | 10 |
| Calcium (mM) | 30 | 30 | 30 | 30 | 30 |
| Phosphate (mM) | 5 | 5 | 5 | 5 | 5 |
| Exenatide (mg/ml) | 0.1 | 0.25 | 0.5 | 1 | 2 |
| Efficiency (%) | 79.7 | 85.8 | 77.4 | 50.2 | 33.3 |

Similar to the result in Example 7, this experiment demonstrates that enhancement of exenatide encapsulation in calcium phosphate nanoparticles by sodium caprate is dependent on exenatide concentration. The encapsulation efficiency was significantly lower at the higher concentrations of exenatide (>1.0 mg/ml), with the optimal exenatide concentrations falling between 0.1 and 0.5 mg/ml. Once again, this is an unexpected and surprising discovery because the encapsulation efficiency of exenatide in the presence of ursodeoxycholate (UDCA) was independent of exenatide concentration (data not shown).

Thus, even though the encapsulation efficiency of biologically active macromolecules such as insulin or exenatide into calcium phosphate nanoparticles can be enhanced to similar degrees by bile salts (e.g., UDCA) and medium chain fatty acids (e.g., caprate), the inventors have unexpectedly discovered that the encapsulation enhancement profiles of these compounds are quite different. The UDCA-induced enhancement is independent of drug concentration, whereas the caprate-induced enhancement exhibits significant concentration dependence. Based on the results obtained to date, the optimal macromolecule concentration for caprate-induced enhancement appears to be between 0.2 and 1.0 mg/ml.

### Example 9

### Insulin Release by UDCA-Containing Nanoparticles

To evaluate insulin release by the calcium phosphate nanoparticles fabricated in Example 6, the nanoparticles were suspended in either a 0.2 M sodium phosphate buffer, pH 9.1, or in a 0.01 N HCl solution, pH 2.0. The nanoparticle concentration was 1.5 mg/ml and the mixtures were shaking at 37° C. for 60 min. The samples were then spun down at 10000 rpm for 10 min to remove the nanoparticles. Supernatant was cleared using a 0.45 μm filter and insulin contents were measured by HPLC. The insulin contents released from UDCA-containing calcium phosphate nanoparticles are shown in Table 12.

TABLE 12

| Sample | 1 | 2 |
|---|---|---|
| Release Buffer | 0.2M sodium phosphate, | 0.01N HCl, pH 2.0 |
| | pH 9.1 | |
| Insulin content | 3.34 | 2.65 |
| (μg/mg) | | |
| Appearance | Clear | Cloudy |

The UDCA-containing nanoparticles were mostly cleared in the 0.2 M sodium phosphate buffer, and insulin release appeared essentially complete. In the 0.01 N HCl solution, however, the particles were not completely dissolved and insulin release was not complete.

### Example 10

### Insulin Release by Caprate-Containing Nanoparticles

To evaluate insulin release by the calcium phosphate nanoparticles fabricated in Example 7, the nanoparticles were suspended in either a 0.2 M sodium phosphate buffer, pH 9.1, or in a 0.01 N HCl solution, pH 2.0. The nanoparticle concentration was 1.5 mg/ml and the mixtures were shaking at 37° C. for 60 min. The samples were then spun down at 10000 rpm for 10 min to remove the particles. The supernatants were cleared using a 0.45 μm and insulin contents were measured by HPLC. The insulin contents released from caprate-containing calcium phosphate particles are shown in Table 13.

TABLE 13

| Sample | 1 | 2 |
|---|---|---|
| Release Buffer | 0.2M sodium phosphate, | 0.01N HCl, pH 2.0 |
| | pH 9.1 | |
| Insulin content | 2.59 | 2.65 |
| (μg/mg) | | |
| Appearance | Cloudy | Clear |

The caprate-containing nanoparticles were mostly cleared in the 0.01 N HCl solution, and insulin release appeared complete. In the 0.2 M sodium phosphate buffer, however, the particles were not completely dissolved yet the insulin content was close to that released from the caprate-containing nanoparticles in the 0.01 N HCl solution.

The results shown in Examples 9 and 10 indicate that UDCA- and caprate-containing calcium phosphate nanoparticles exhibit different insulin release behavior. The UDCA-containing nanoparticles show more efficient particle dissolution and more complete insulin release in a high pH (0.2 M sodium phosphate, pH 9.1), whereas the caprate-containing nanoparticles show more efficient particle dissolution at a low pH (0.01 N HCl, pH 2.0). Surprisingly, the insulin content released by the caprate-containing nanoparticles appeared independent of pH in this study.

1. A plurality of particles comprising:
a. a plurality of calcium phosphate core nanoparticles;
b. a GLP-1 agonist encapsulated in the core nanoparticles; and
c. a co-precipitating agent comprising a bile salt encapsulated in the core nanoparticles;
whereas the presence of the bile salt enhances encapsulation efficiency of the GLP-1 agonist into the core nanoparticles relative to calcium phosphate core nanoparticles that do not comprise the bile salt.

2. The particles of claim 1, wherein the GLP-1 agonist is exenatide or a physiologically acceptable salt or derivative thereof

3. The particles of claim 1, wherein the core nanoparticles have an average diameter of less than 300 nm.

4. The particles of claim 1, wherein the bile salt is selected from the group consisting of a cholate, a deoxycholate, a taurocholate, a glycocholate, a taurodeoxycholate, an ursodeoxycholate, a tauroursodeoxycholate, a chenodeoxycholate, and a combination thereof.

5. A pharmaceutical composition comprising the particles of claim 1 and a pharmaceutically acceptable carrier.

6. The pharmaceutical composition of claim 5, wherein the composition is in the form of a capsule, a tablet, a sphere, or a powder.

7. The pharmaceutical composition of claim 6, wherein the composition further comprises an enteric coating.

8. The pharmaceutical composition of claim 5, further comprising an absorption enhancer.

9. The pharmaceutical composition of claim 8, wherein the absorption enhancer is a medium chain fatty acid salt.

10. The pharmaceutical composition of claim 9, wherein the medium chain fatty acid salt is selected from the group consisting of a caproate, a caprylate, a pelargonate, a caprate, a laurate, a myristate, and a combination thereof.

11. A method of treating a subject in need of a GLP-1 agonist treatment, said method comprising administering to the subject a therapeutically effective amount of the pharmaceutical composition of claim 5.

12. The method of claim 11, wherein the pharmaceutical composition is administered via the oral route.

13. The method of claim 11, wherein the pharmaceutical composition is administered to a mucosal surface.

14. A pharmaceutical composition comprising:
i. a plurality of particles comprising:
a. a plurality of calcium phosphate core nanoparticles;
b. a GLP-1 agonist encapsulated in the core nanoparticles; and
c. a co-precipitating agent comprising a fatty acid salt encapsulated in the core nanoparticles; and
ii. an absorption enhancer,
wherein the presence of the fatty acid salt enhances encapsulation efficiency of the GLP-1 agonist into the core nanoparticles relative to calcium phosphate core nanoparticles that does not comprise the fatty acid salt.

15. The pharmaceutical composition of claim 14, wherein the core nanoparticles have an average diameter of less than 300 nm.

16. The pharmaceutical composition of claim 14, wherein the fatty acid salt is selected from the group consisting of a caproate, a caprylate, a pelargonate, a caprate, a laurate, a myristate, and a combination thereof.

17. (canceled)

18. (canceled)

19. The pharmaceutical composition of claim 14, wherein the GLP-1 agonist is exenatide or a physiologically acceptable salt or derivative thereof.

20. The pharmaceutical composition of claim 14, further comprising a pharmaceutically acceptable carrier.

21. The pharmaceutical composition of claim 20, wherein the composition is in the form of a capsule, a tablet, a sphere, or a powder.

22. The pharmaceutical composition of claim 21, wherein the composition further comprises an enteric coating.

23. (canceled)

24. The pharmaceutical composition of claim 14, wherein the absorption enhancer is a medium chain fatty acid salt.

25. The pharmaceutical composition of claim 24, wherein the medium chain fatty acid salt is selected from the group consisting of a caproate, a caprylate, a pelargonate, a caprate, a laurate, a myristate, and a combination thereof.

26. A method of treating a subject in need of a GLP-1 agonist treatment, said method comprising administering to the subject a therapeutically effective amount of the pharmaceutical composition of claim 14.

27. The method of claim 26, wherein the pharmaceutical composition is administered via the oral route.

28. The method of claim 26, wherein the pharmaceutical composition is administered to a mucosal surface.

29. A method of making a plurality of calcium phosphate particles, said method comprising:
a. contacting an aqueous solution of a calcium salt with an aqueous solution of a phosphate salt in the presence of a co-precipitating agent comprising a fatty acid salt;
b. mixing the resulting solution in step a) until a calcium phosphate particle of a desired size is obtained; and
c. recovering the calcium phosphate particles.

30. The method of claim 29, wherein the fatty acid salt is selected from the group consisting of a caproate, a caprylate, a pelargonate, a caprate, a laurate, a myristate, and a combination thereof.

31. The method of claim 29, wherein the calcium salt has a concentration ranging from about 5 mM to about 200 mM.

32. The method of claim 29, wherein the phosphate salt has a concentration ranging from about 5 mM to about 200 mM.

33. The method of claim 29, further comprising adding a biologically active macromolecule into the aqueous solution of the phosphate salt or the aqueous solution of the calcium salt before contacting the aqueous solution of the calcium salt with the aqueous solution of the phosphate salt in the presence of the co-precipitating agent comprising a fatty acid salt, whereby the calcium phosphate particles are co-crystallized with the biologically active macromolecule.

34. The method of claim 33, wherein the biologically active macromolecule is selected from the group consisting of a protein, a peptide, a polysaccharide, a nucleic acid, a lipid, and a carbohydrate.

35. The method of claim 34, wherein the biologically active macromolecule is selected from the group consisting of a GLP-1 agonist, an insulin, an erythropoietin, an interferon, a growth hormone, a PTH, a calcitonin, a leuprolide, and a derivative thereof.

36. The method of claim 35, wherein the GLP agonist is exenatide or a physiologically acceptable salt or derivative thereof.
